(19) United States
(12) Patent Application Publication
(10) Pub. No.: US 2023/0234132 A1
(43) Pub. Date: Jul. 27, 2023

(54) SIMULATION SYSTEM FOR SELECTING AN ALLOY, AND A PRODUCTION PROCESS FOR A WORKPIECE TO BE PRODUCED HAVING AMORPHOUS PROPERTIES

(71) Applicant: Heraeus Amloy Technologies GmbH, HANAU (DE)

(72) Inventors: Hans Jürgen WACHTER, Karlstein (DE); Eugen MILKE, Karlstein (DE); Hamed SHAKUR SHAHABI, Hanau (DE)

(21) Appl. No.: 17/999,755

(22) PCT Filed: May 25, 2021
(86) PCT No.: PCT/EP2021/063838

§ 371 Date: Nov. 23, 2022

(30) Foreign Application Priority Data
May 28, 2020 (EP) 20177036.9

(51) Int. Cl. B22F 10/30; B22F 3/00; B22F 10/80

(52) U.S. Cl. CPC B22F 10/30; B22F 3/006; B22F 10/80; B33Y 70/00

(57) ABSTRACT

Simulation system for selecting an alloy and a production process for a workpiece to be produced having amorphous properties, wherein the system includes : an input unit, for inputting a requirements profile for the workpiece to be produced, at least one memory unit, to store information data, wherein the information data specifies information concerning physical and/or chemical and/or mechanical properties of a number of alloys for manufacturing workpieces having amorphous properties and information concerning production processes, an analysis unit, to simulate a number of workpieces according to the requirements profile and the information data to create simulation data, to assess the simulated workpieces on the basis of the simulation data and the requirements profile, to select an alloy and a production process for the workpiece to be produced from assessment, and an output unit, to output the selected alloy and the selected production process.

The invention relates to a simulation system and a method for selecting an alloy and a manufacturing method for a workpiece to be manufactured having amorphous properties, a computer-readable storage medium, a manufacturing plant for manufacturing a workpiece having amorphous properties, and a control method.

Amorphous metals are a new class of materials which have physical properties or combinations of properties that cannot be realized in other materials.

The term “amorphous metals” refers to metal alloys that do not have a crystalline structure but an amorphous structure on the atomic level. The amorphous atomic arrangement, which is unusual for metals, results in unique combinations of physical properties. Amorphous metals are generally harder, more corrosion-resistant and stronger than conventional metals, and are at the same time highly elastic. The absence of grain boundaries results in less chemical attack surface, and therefore metallic glasses are less susceptible to corrosion.

Metallic glasses, as amorphous metals are also called, have been the subject of extensive research ever since they were discovered at the California Institute of Technology. Over the years, it was possible to continuously improve the processability and the properties of this material class. While the first metallic glasses were simple, binary alloys (composed of two components), the production of which required cooling rates in the range of 106 Kelvin per second (K/s), newer, more complex alloys can be converted into the glassy state at significantly lower cooling rates in the range of a few K/s. This has a significant influence on process management and the workpieces that can be produced. The cooling rate from which crystallization of the melt ceases to apply and the melt solidifies in the glassy state is referred to as the critical cooling rate. The critical cooling rate is a system-specific variable which is strongly dependent on the composition of the melt and which moreover defines the maximum achievable component thicknesses. Considering that the thermal energy stored in the melt has to be removed sufficiently quickly by the system, it is clear that only workpieces with a small thickness can be produced from systems with high critical cooling rates. At first, metallic glasses were therefore usually produced by melt spinning. In this case, the melt is stripped onto a rotating copper wheel and solidifies in a glass-like manner in the form of thin strips or films with thicknesses in the range of a few hundredths to tenths of a millimeter. As a result of the development of new, complex alloys with significantly lower critical cooling rates, it is increasingly possible to use other production methods. Today’s bulk-glass-forming metal alloys can already be converted into the glassy state by casting a melt into cooled copper molds. In this case, the realizable component thicknesses are in the range of a few millimeters to centimeters, depending on the alloy. Alloys of this kind are referred to as bulk metallic glasses (BMG). Nowadays, a large number of such alloy systems are known.

The subdivision of bulk metallic glasses usually takes place on the basis of the composition, wherein the alloy element having the highest proportion by weight is referred to as the base element. The existing systems comprise, for example, noble metal-based alloys such as gold-, platinum-, and palladium-based bulk metallic glasses, early transition metal-based alloys such as titanium- or zirconium-based bulk metallic glasses, late transition metal-based systems based on copper, nickel or iron, but also systems based on rare earths, for example neodymium or terbium.

Bulk metallic glasses typically have the following properties compared to traditional crystalline metals:
higher specific strength, which enables, for example, thinner wall thicknesses,
higher hardness, whereby the surfaces can be particularly scratch-resistant,
much higher elastic stretchability and resilience,
thermoplastic formability and
higher corrosion resistance.

Due to their advantageous properties, such as high strength and the absence of solidification shrinkage, metallic glasses, in particular bulk metallic glasses, are very interesting construction materials which are suitable in principle for the production of components in series production methods such as injection molding, without further processing steps being mandatory after shaping. In order to prevent crystallization of the alloy during cooling from the melt, a critical cooling rate must be exceeded. However, the greater the volume of the melt, the slower the cooling of the melt (with otherwise unchanged conditions). If a certain sample thickness is exceeded, crystallization occurs before the alloy can solidify amorphously.

In addition to the excellent mechanical properties of metallic glasses, unique processing options also result from the glassy state. Thus, metallic glasses can be shaped not only by metallurgical melting processes, but also by thermoplastic molding at comparatively low temperatures in a manner analogous to thermoplastic materials or silicate glasses. For this purpose, the metallic glass is first heated above the glass transition point to then behave like a highly viscous liquid which can be formed at relatively low forces. After forming, the material is again cooled below the glass transition temperature.

When processing amorphous metals, the natural crystallization is prevented by rapid cooling (freezing in the molten state) of the melt so that the atoms lose their mobility before they can assume a crystal arrangement. Many properties of crystalline materials are influenced or determined by faults in the atomic structure, i.e., by so-called lattice defects (gaps, shifting, grain boundaries, phase boundaries, etc.).

As a result of the rapid cooling, the shrinkage of the material is reduced so that more precise component geometries can be achieved in amorphous metals. Plastic deformation only takes place with elongations above 1.8%. In comparison, crystalline metallic materials show irreversible deformation at significantly lower elongations (<0.5%). Moreover, the combination of high yield strength and high elastic elongation results in a high elastic energy storage capacity.

However, the thermal conductivity of the material used sets a physical limit to the cooling rate, since the heat contained in the component must be released to the environment via the surface. This leads to limitations in the manufacturability of components and in the applicability of production methods.

Various methods for producing workpieces from amorphous metals are known. It is possible, for example, to produce workpieces using additive manufacturing methods such as 3D printing. The amorphous properties of the workpiece can be ensured by adjusting the process parameters such as the scan speed, the energy of the laser beam or the pattern to be scanned.

One advantage of the additive manufacturing technique is that in principle any conceivable geometry can be realized. Furthermore, it may be advantageous that, in the case of additive manufacturing methods, no separate cooling process is necessary, since effective cooling can be ensured by the layer-by-layer production of the workpiece and by adjusting the size of the melt pool via the laser energy and scan path of the laser.

A disadvantage of additive manufacturing methods is the low build-up rates at the time of the application, especially for large-dimensioned workpieces. Furthermore, high-purity powder material must be used as a starting material for the additive manufacturing process for some applications, i.e. for certain workpieces to be manufactured. If impurities are present in the material, crystallization can occur at the locations of the impurities, resulting in non-amorphous metal, which can lead to a deterioration in the mechanical and chemical properties. It may be necessary to finish the surface of the workpiece due to the impurities, which is complicated. In addition, additive manufacturing always results in a certain roughness on the surface of the workpiece, so that in most cases it has to be finished by grinding or milling.

A further production possibility is injection molding. In this case, workpiece weights in the range of 80-120 g or greater can be realized at the time of the application. The material to be used is usually heated within approximately 10-60 seconds to approximately 900-1100° C. by induction heating and is homogenized.

After heating, the molten material is pressed into a mold by means of a punch. It is important for the material properties that when the mold is completely filled with material, the material within the mold should have a temperature above the material melting point throughout. In order to achieve amorphous material properties, the liquid material within the mold must subsequently be cooled rapidly to below the glass transition temperature.

The possible geometries in injection molding are limited to wall thicknesses of 0.3-7.0 mm due to the cooling rate of the material. In the case of larger wall thicknesses, the cooling rate is too low, so that crystalline structures form before the material has cooled to below the glass transition temperature. With smaller wall thicknesses, the material cools too quickly depending on the length to be filled and solidifies before the mold is completely filled.

To ensure in advance during the construction, dimensioning, selection of the alloy material, selection of the production method or the like that the amount of heat supplied to the material can be released sufficiently quickly to the environment, the cooling behavior can be simulated and analyzed.

DE 10 2015 110 591 A1 describes, for example, a device and a manufactured product for predicting material properties of a cast aluminum-based component. In this case, a computer-based system comprises numerous calculation modules which interact with one another in terms of programming in such a way that the modules provide performance features of the material when they receive data corresponding to the cast aluminum-based component.

It is disadvantageous here that the described device is only designed to select an alloy and not additionally to select a manufacturing method. As already explained in detail above, a selection of a suitable alloy and a suitable manufacturing method is difficult for a user without specific technical knowledge of amorphous metals.

EP3246831 describes, based on numerical methods, a scalable and predictive 3D printing simulation for the production of complex components, wherein primarily the printing profile, printing time and cooling capacity are simulated on the basis of localized heating effects. A manufacturability analysis of the component, in particular with respect to its molten state and subsequent cooling, is not carried out.

Furthermore, DE102006047806 discloses simulating a model of hot forming a metal blank from a convertible steel material with the aid of the finite element method. With hot-forming simulation, not only are the mechanical and physical properties of the steel material to be formed taken into account, but material data which are incorporated into the method in the form of a time-temperature conversion dataset of the specific steel material are taken into account in the context of a complex thermo-mechanically coupled simulation. In this way, on the basis of the relevant phase composition, the determined temporary local mechanical property values can be transferred to a failure model for improving the component prognosis and for process optimization.

DE102006047806 thus describes a simulation method for modeling hot forming of a metal blank from a convertible steel material with the aid of the finite element method. In this case, temporary local mechanical properties, for example the hardness and the physical properties of the metal plate during and after the end of the hot-forming simulation on the basis of the local and temporary phase composition of the steel material, are the focus.

Another example is known from WO2018182513, which describes a computer-implemented method for assessing geometric changes of an object to be produced by an additive manufacturing process, wherein during this additive manufacturing process a crystallizable material is converted from a powder to a bulk form and in the process the object is formed from the bulk form.

The method of WO2018182513 comprises:
i. providing a simulation domain comprising a finite element model of the object embedded into a simulated cake of the powder; wherein the finite element model comprises finite elements of the object and finite elements of the simulated cake of the powder;
ii. associating thermal properties of the bulk crystal resinable material with each finite element of the object;
iii. associating the thermal properties of the powder crystallizable material with each finite element of the simulated cake of the powder;
iv. associating a simulated first temperature with each finite element;
v. performing a finite element analysis of the finite element model under a simulated cooling condition, wherein the simulated cooling condition comprises applying a simulated second temperature to at least one boundary of the simulation area, wherein the simulated second temperature is lower than the simulated first temperature;

In this case, the finite element analysis of WO2018182513 comprises the following:
i. determining a simulated crystalline volume fraction of the simulated bulk crystallizable material for each finite element of the object;
ii. determining a simulated thermal expansion coefficient for each finite element of the object as a function of the simulated crystalline volume fraction, the thermal expansion coefficient (<3 ~ 4) of a crystalline phase of the crystallizable material, and the thermal expansion coefficient of an amorphous phase of the crystallizable material;
iii. performing the finite element analysis until an equilibrium state is reached.

WO2018182513 describes a computer-implemented method for assessing geometric changes of a component to be produced by an additive manufacturing process. Here, a crystallization-related change in volume is the focus.

From the described disadvantages of the prior art, the object is to provide a user with a simulation system for the simple selection of an alloy and of a manufacturing method for a workpiece to be manufactured having amorphous properties.

The object is achieved according to the invention by a simulation system having the features of claim 1. Advantageous embodiments, developments and variants form the subject matter of the dependent claims.

In particular, the object is achieved by a simulation system for selecting an alloy and a manufacturing method for a workpiece to be manufactured having amorphous properties, the system comprising an input unit for inputting a requirements profile for the workpiece to be manufactured.

Furthermore, the system comprises at least one memory unit which is designed to store information data, wherein the information data specify information concerning physical and/or chemical and/or mechanical properties a plurality of alloys for the production of workpieces having amorphous properties. For simplicity, only one memory unit is mentioned in the context of the following explanations, in a non-limiting manner. However, it is also possible for a plurality of memory units or a memory unit with a plurality of partitions or modules to be provided.

Furthermore, the information data also provide information concerning manufacturing methods, in particular for manufacturing such workpieces.

In addition, the simulation system comprises an analysis unit. The analysis unit is designed to simulate a plurality of workpieces according to the requirements profile and the information data to create simulation data. Furthermore, the analysis unit is designed to assess the simulated workpieces on the basis of the simulation data and the requirements profile.

The analysis unit is further designed to select an alloy and a manufacturing method for the workpiece to be manufactured on the basis of the assessment. This selection is preferably carried out in such a way that an alloy and a manufacturing method are selected of which the simulated workpiece - that is to say a workpiece which has been simulated from this alloy with a manufacturing method stored in the memory unit - substantially corresponds or ideally completely corresponds to that of the workpiece to be produced or its requirements profile. Within the scope of this application, an identity can substantially mean with a deviation of less than or equal to 1%, 5%, 10%, 20%, 25% or 30% from a mass, a volume or generally a parameter of the workpiece to be manufactured.

The simulation system also comprises an output unit which is designed to output the selected alloy and the selected manufacturing method for a user. That is to say, the output unit communicates the selected alloy and/or the selected manufacturing method to the user.

The simulation system according to the invention thus provides a selection of an alloy and of a manufacturing method for a workpiece to be manufactured having amorphous properties, by means of which a suitable alloy and a suitable manufacturing method for the workpiece can be specified without expert knowledge of the user. The requirements profile can thus be met in the best possible way.

In one embodiment, the analysis unit can comprise a calculation unit which can be designed to calculate properties of the workpiece to be manufactured which are specified by the requirements profile. These can in particular be properties which are not directly apparent from the requirements profile of the workpiece to be manufactured, but which are important for the selection of a suitable alloy and of a suitable manufacturing method. As a result, due to a greater information density, the simulation and the subsequent selection of a suitable alloy and of a suitable manufacturing method are made more precise.

In one embodiment, the analysis unit can comprise a first simulation unit which can be designed to simulate, depending on the alloy and according to the requirements profile and the calculated properties, a mechanical load on the workpiece to be manufactured and to add information concerning the simulated mechanical load to the simulation data. The mechanical load can be understood to mean, for example, a (required) bending and/or torsional resistance or load on the workpiece to be manufactured. Alternatively, the mechanical load can also be understood as mechanical properties such as a stiffness of the workpiece to be manufactured.

In a further embodiment, the analysis unit can comprise a second simulation unit which can be designed to simulate, depending on the alloy and according to the requirements profile and the information data, chemical properties of the workpiece to be manufactured and to add information concerning the simulated chemical properties to the simulation data. The chemical properties can be, for example, a corrosion behavior or a media resistance, in particular when the workpiece to be manufactured will be exposed to acids and/or bases.

Alternatively or additionally, in a further embodiment, the analysis unit can comprise a third simulation unit. The third simulation unit can be designed to simulate manufacturing of the workpiece by means of the manufacturing method contained in the at least one memory unit, according to the requirements profile. In this case, as already mentioned above, workpieces are simulated which are produced in a simulated manner with the manufacturing methods contained on the at least one memory unit. The information concerning the simulated manufacturing can also be added to the simulation data, as in the above two embodiments.

The advantage here can be seen in increasing the information data and making it more precise, and thus in increasing the quantity in relation to the information data, whereby the simulation system has more information available for the selection of the suitable alloy and of the suitable manufacturing method and thus a more accurate selection can take place.

The first, second and third simulation units can be designed as a single unit or can be divided logically. However, they can be designed as a single data structure or as a function or a plurality of functions of a program.

The requirements profile can preferably specify geometric and/or mechanical and/or chemical properties of the workpiece to be manufactured. For example, the geometric properties can be understood to mean a dimension and/or a weight of the workpiece to be manufactured. In particular the weight of the workpiece to be manufactured has an influence on a suitable manufacturing method, for example. While, for example, workpieces with a weight between 80 gr and 100 gr can preferably be produced by means of an injection-molding method, workpieces with a mass deviating from this above-mentioned weight range are expediently produced by means of a 3D printing method.

Some manufacturing methods and/or some alloys are also preferred for complex geometric structures of the workpiece to be manufactured, while other alloys and/or manufacturing methods may be inexpedient for some geometric structures. However, the mechanical properties can preferably be understood to mean, non-exhaustively, the mechanical properties of the workpiece to be manufactured already described above.

The chemical properties of the workpiece to be manufactured can also be understood as the chemical properties already mentioned above. However, this can also be understood as meaning, for example, a quality of the alloy to be selected, i.e., for example, specifically an oxygen content of the relevant alloy. This chemical property is expediently to be considered in relation to a size of the workpiece to be manufactured, since even higher demands on the quality of the starting element are preferably set in the case of large workpieces to be manufactured (for example 2-6 mm in the case of Zr-based alloys). In the case of smaller workpieces to be manufactured (e.g. < 2 mm in the case of Zr-based alloys), the quality and especially the oxygen content of the alloy may sometimes be negligible with regard to cost efficiency. Furthermore, however, the chemical property can also be understood to mean a biocompatibility of the alloy, which is reflected in particular in the medical field or in medical applications. For instance, in particular copper-free alloys have proven advantageous in the medical technology sector. With regard to the corrosion behavior already mentioned above, but also with regard to so-called metal ion release, for example with perspiration, in particular zirconium-, titanium- or platinum-based alloys have proven advantageous.

In one embodiment, the information data can thus specify information concerning at least one physical and/or chemical and/or mechanical properties. In particular, these are the property groups listed below:
thermal properties of the alloy,
media resistance of the alloy,
chemical properties,
amorphicity on the basis of a degree of contamination (oxygen content),
load-dependent aging phenomena and/or
cooling behavior of the alloy.

In particular the last-mentioned property, i.e. the cooling behavior of the alloy, preferably relates to the manufacturing method to be selected, while the other properties/property groups listed above are primarily aimed at the workpiece to be manufactured. The memory unit can be designed, for example, as a database. The aforementioned properties can then be storable or stored as a data record in the database, for example.

In one embodiment, the information data can additionally contain information concerning the manufacturing steps of the manufacturing methods. In this case, the information can be, in particular a punch speed and/or a starting temperature of the alloy to be processed. However, other process information concerning the manufacturing steps of the manufacturing method can also be part of the information. The selection in particular of a suitable manufacturing method is thus further improved.

In one embodiment, the analysis unit can be designed to make, according to the requirements profile, a preselection alloy stored within the memory unit and or from manufacturing methods stored within the memory unit. In addition, a suitable alloy and/or a suitable manufacturing method can be selected using this preselection. For example, chemical requirements such as the above-mentioned biocompatibility of the alloy, but also mechanical requirements, for example abrasion resistance, hardness and electrical requirements and/or also magnetic requirements can be taken into account in the preselection.

The preselection can thus be understood to mean that, on the basis of these aforementioned properties, certain alloys and/or manufacturing methods are already excluded without a simulation because unsuitable alloys or manufacturing methods are no longer considered for later selection, and therefore simulation effort is minimized.

In one embodiment, the analysis unit can be designed to generate a data pair by associating the selected alloy and the selected manufacturing method with the requirements profile. In other words, a selected alloy and a selected manufacturing method can be associated with a requirements profile, and this association can be stored in the form of a data pair in the memory unit. Furthermore, in this embodiment, the analysis unit can be designed to specify an alloy associated with the requirements profile and an associated manufacturing method when a requirements profile stored in the memory unit is input by the user. Thus, if such a requirements profile is input, a simulation can be dispensed with, and a suitable alloy and a suitable method are proposed and output immediately.

The advantage here can be seen in the significantly reduced simulation effort. Likewise, the simulation system and a selection of a suitable alloy and/or of a suitable manufacturing method can be accelerated on the one hand and simplified on the other hand.

In the context of this application, a method for selecting an alloy and a manufacturing method for a manufacturing workpiece having amorphous properties is also disclosed and claimed, the method comprising the following steps:
inputting a requirements profile of the workpiece to be manufactured, in particular by means of an input unit,
calculating mechanical and/or chemical and/or physical parameters, and comparing the calculated parameters with stored information data which contain information concerning physical and/or chemical and/or mechanical properties of a plurality of alloys for producing workpieces having amorphous properties,
simulating a plurality of workpieces according to the requirements profile and the information data,
creating simulation data on the basis of the simulations,
assessing the simulated workpieces on the basis of the simulation data and the requirements profile,
selecting an alloy and a manufacturing method for the workpiece to be manufactured on the basis of the assessment,
outputting the selected alloy and the selected manufacturing method.

In one embodiment of the method, properties of the workpiece to be manufactured which are specified by the requirements profile can be calculated.

In one embodiment, an alloy-dependent simulation of a mechanical load on the workpiece to be manufactured can take place. This can take place according to the requirements profile and the calculated properties. Furthermore, information concerning the simulated mechanical load can be added to the simulation data.

In one embodiment, chemical properties of the workpiece to be manufactured can additionally be simulated depending on the alloy and according to the requirements profile and the information, and information concerning these simulated chemical properties can be added to the simulation data.

Alternatively or additionally to the aforementioned embodiments, manufacturing of the workpiece can be simulated according to the requirements profile by means of the stored manufacturing methods. Information concerning the simulated manufacturing can then be added to the simulation data.

In one embodiment, by means of the information data, information concerning at least one physical and/or chemical and/or mechanical properties can be specified, in particular selected from:
thermal properties of the alloy,
media resistance of the alloy,
chemical properties,
amorphicity on the basis of a degree of contamination,
load-dependent aging phenomena and/or
cooling behavior of the alloy.

This ensures that at least the properties which are important for selecting a suitable alloy and/or a suitable method are specified and thus used as the basis for the method for selection.

In a further embodiment, a preselection from stored alloys and/or stored manufacturing methods can be made according to the requirements profile. Furthermore, in this embodiment, a selection of an alloy and/or a manufacturing method can take place on the basis of the preselection.

In one embodiment, a data pair can be generated by associating the selected alloy and the selected manufacturing method with the requirements profile. The generated data pair can then be stored in the memory unit.

If a requirements profile which is already stored on the memory unit is now input, a simulation is preferably not started, but rather there is an output of an alloy associated with the stored requirements profile and a manufacturing method associated with the requirements profile.

The object is also achieved according to the invention by a computer-readable storage medium which contains instructions that cause at least one processor to implement a method when the method is executed by the at least one processor. The method is the method described above for selecting an alloy and a manufacturing method for a workpiece to be manufactured having amorphous properties. Furthermore, a manufacturing plant for manufacturing a workpiece having amorphous properties is disclosed and claimed, wherein the manufacturing plant comprises a simulation system for selecting an alloy and a manufacturing method for the workpiece to be manufactured. The simulation system is in particular the simulation system already described above in the context of this application. Furthermore, the manufacturing plant comprises a manufacturing unit which is designed to manufacture a workpiece using the simulation system.

In one embodiment of the manufacturing plant, the manufacturing unit can be designed as an injection-molding device or as an additive manufacturing device. The additive manufacturing device can be understood here to mean, for example, a 3D printing device. As a result, the manufacturing plant can be adapted to different requirements, in particular with regard to a size of the workpieces to be manufactured.

In addition, a control method for controlling a manufacturing plant for manufacturing a workpiece having amorphous properties is disclosed and claimed. The control method is used in particular to control the manufacturing plant described above. In this case, the manufacturing plant is operated with an alloy and a manufacturing method, wherein both the alloy and the manufacturing method are selected, in particular with the method described above for selecting an alloy and a manufacturing method.

The advantages and preferred embodiments mentioned with respect to the simulation system can be transferred analogously to methods for selecting an alloy and/or a manufacturing method, to the computer-readable storage medium, and to the manufacturing plant and the control method thereof, and vice versa.

The simulation system and the manufacturing plant can also be arranged so as to be spatially separate from one another. In this case, the simulation system and the manufacturing plant can then communicate with one another via a communication network, for example the Internet. The output of the suitable alloy and the suitable manufacturing method and/or the input of the requirements profile can take place, for example, on a website or via a programming interface, such as an API.

The invention is explained in more detail below with reference to an exemplary embodiment. In the drawings:

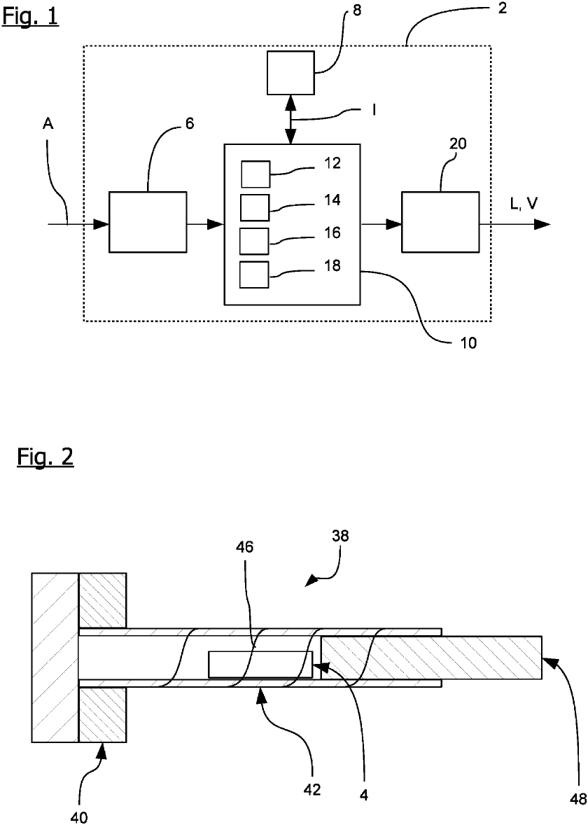

FIG. 1 shows a schematic simulation system for selecting an alloy and a manufacturing method for a workpiece to be manufactured having amorphous properties;

FIG. 2 shows a schematic illustration of a manufacturing unit, and

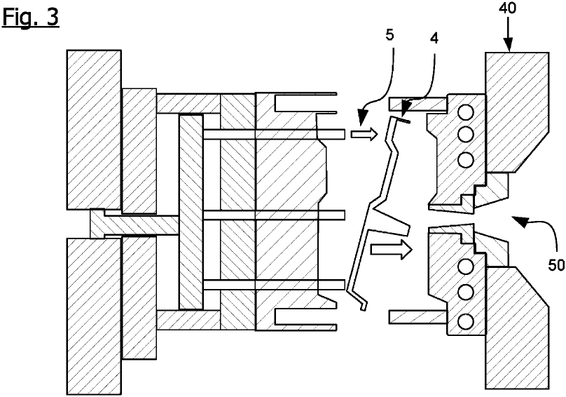

FIG. 3 shows a schematic illustration of a tool.

FIG. 1 shows a schematically illustrated simulation system 2. The simulation system 2 is designed to select an alloy and a manufacturing method for a workpiece 4 to be manufactured (cf. FIG. 4) having amorphous properties.

For this purpose, the simulation system 2 according to FIG. 1 comprises an input unit 6 which is used to input a requirements profile A for the workpiece 4 to be manufactured. This makes it possible for a user to provide the required properties of the workpiece 4 to be manufactured in the form of the requirements profile A to the simulation system 2 or to feed the simulation system 2 with the requirements profile A.

Furthermore, the simulation system 2 comprises at least one memory unit 8 which is designed to store information data I, wherein the information data I specify information concerning physical and/or chemical and/or mechanical properties of a plurality of alloys L for producing workpieces 4 and information concerning manufacturing methods V. For this purpose, the memory unit 8 can have, for example, a plurality of partitions and/or can be designed in multiple parts, i.e. composed of a plurality of sub-memory units. Furthermore, the memory unit 8 is communicatively and bidirectionally connected to an analysis unit 10.

In this case, the analysis unit 10 is designed to simulate a plurality of workpieces 4 according to the requirements profile A and the information data I. This is used to create simulation data. Furthermore, the analysis unit 10 is configured to assess the simulated workpieces on the basis of the simulation data and the requirements profile A, before of an alloy L and a manufacturing method V which are particularly suitable with regard to the requirements profile A of the workpiece 4 to be manufactured are selected on the basis of the assessment.

In order to realize the above method steps, the analysis unit 10 comprises a calculation unit 12, by means of which properties of the workpiece to be manufactured that are specified by the requirements profile A are calculated. Furthermore, the analysis unit 10 comprises a first simulation unit 14. By means of the first simulation unit 14, a simulation of a mechanical load on the workpiece 4 to be manufactured takes place on the basis of and according to the requirements profile A and the calculated properties. Subsequently, the information concerning the simulated mechanical load is added to the simulation data.

Analogously, the analysis unit 10 also comprises a second simulation unit 16 and a third simulation unit 18. A simulation of chemical properties of the workpiece 4 to be manufactured is carried out by means of the second simulation unit 16 according to the requirements profile A and the information data I. By means of the third simulation unit 18, manufacturing of the workpiece 4 is simulated by means of the manufacturing methods V contained in the memory unit 8. Both the information concerning the chemical properties, which is provided by the second simulation unit 16, and the information concerning the simulated manufacturing, which is generated by the third simulation unit 18, are subsequently added to the simulation data and used for a selection of a suitable alloy L and of a suitable manufacturing method V as part of the assessment by the analysis unit 10. The selection and the assessment are carried out in such a way that the alloy L and the manufacturing method V which fulfill or at least substantially fulfill the properties of the workpiece 4 to be manufactured, defined by the requirements profile A, are selected. If a plurality of alloys L and/or a plurality of manufacturing methods V come into consideration, the analysis unit 10 selects the alloy L and the manufacturing method V which are most suitable with regard to higher-level preferences, for example with regard to cost efficiency.

After the selection of the suitable alloy L and the suitable manufacturing method V by the analysis unit 10, the selected alloy L and the selected manufacturing method V are output by an output unit 20. The output unit 20 can, for example, be an optical output unit 10 in which the output is effected on a screen.

According to one embodiment, the analysis unit 10 is designed to generate a data pair by associating the selected alloy L and the selected manufacturing method V with the requirements profile. In this case, the generated data pair is stored in the memory unit 8. If a requirements profile A stored in the memory unit 8 is now input, an alloy L associated with the requirements profile A and a manufacturing method V associated with the requirements profile A are specified, without a simulation taking place.

In one embodiment, it is conceivable for the simulation system 2 to have an artificial neural network to optimize the selection of the suitable alloy L and/or the suitable manufacturing method V.

FIG. 2 shows a schematic illustration of a manufacturing unit 38, which is designed as an AMM (amorphous metal) injection-molding plant. The manufacturing unit 38 comprises a mold in the tool 40 and a melting chamber 42. The melting chamber 42 is supplied with a solid alloy segment of an amorphously solidifying alloy (blank) 44 by a robot and is placed centrally in an induction coil 46. The blank 44 (“4” instead of “44” in the figure) is heated within the melting chamber 42 by means of a heating element, in particular an induction field which is generated by the induction coil 46. The blank 44 is a solid alloy segment of an amorphously solidifying alloy. The alloy segment 44 comprises, for example, a certain amount of palladium, platinum, zirconium, titanium, copper, aluminum, magnesium, niobium, silicon and/or yttrium.

The blank 44 is melted by the heating element or the induction coil 46, so that it is present in molten form. Preferably, the blank 44 is heated to a temperature of 1050° C. The molten material is injected into the tool 40 by a plunger 48.

FIG. 3 shows the schematic structure of an injection-molding tool 40. The molding chamber 52 is filled with a melt by means of one or a plurality of openings 50 leading into a molding chamber 52 of the tool 40. The molding chamber 52 is designed as a negative mold of the workpiece 4 to be produced. In the exemplary embodiment of FIG. 3, it is provided that an opening 50 can be used to guide liquid material into the molding chamber 52. It can be advantageous to use a plurality of sprues for filling the molding chamber 52 in order to achieve a uniform temperature distribution and to reduce turbulence of the melt. A uniform temperature distribution and a small number of turbulences lead to a better cooling operation, to homogeneous cooling and thus to uniform amorphous material properties.

The liquid material must rapidly cool down within the molding chamber 52 in order to prevent crystallization. The cooling of the liquid material depends greatly on the geometry of the component or workpiece 4 to be produced.

The invention is not limited to the exemplary embodiment described above. Rather, other variants of the invention can also be derived therefrom by a person skilled in the art without departing from the subject matter of the invention. In particular, all individual features described in connection with the exemplary embodiment can also be combined with one another in another way without departing from the subject matter of the invention.

List of reference signs

| | |
|---|---|
| 2 | Simulation system |
| 4 | Workpiece |
| 6 | Input unit |
| 8 | Memory unit |
| 10 | Analysis unit |
| 12 | Calculation unit |
| 14 | First simulation unit |
| 16 | Second simulation unit |
| 18 | Third simulation unit |
| 20 | Output unit |
| 22 | Artificial neural network |
| 24 | Input data |
| 26 | Feature detectors |
| 28 | First fold |
| 38 | Manufacturing unit |
| 40 | Tool |
| 42 | Melting chamber |
| 44 | Blank of an amorphously solidifying alloy |
| 46 | Induction coil |
| 48 | Plunger |
| 50 | Opening |
| 52 | Molding chamber |
| A | Requirements profile |
| I | Information data |
| L | Alloy |
| V | Manufacturing method |

1. A simulation system for selecting an alloy and a manufacturing method for a workpiece to be manufactured having amorphous properties, the system comprising:
an input unit for inputting a requirements profile for the workpiece to be manufactured,
at least one memory unit which is designed to store information data, wherein the information data specify information concerning physical and/or chemical and/or mechanical properties of a plurality of alloys for producing workpieces having amorphous properties and information concerning manufacturing methods, and wherein the requirements profile specifies geometric and/or mechanical and/or chemical properties of the workpiece to be manufactured,
an analysis unit which is designed
to simulate a plurality of workpieces according to the requirements profile and the information data to create simulation data,
to assess the simulated workpieces on the basis of the simulation data and the requirements profile,
to select an alloy and a manufacturing method for the workpiece to be manufactured on the basis of the assessment,
an output unit which is designed to output the selected alloy and the selected manufacturing method.

2. The simulation system according to claim 1, wherein the analysis unit comprises a first simulation unit which is designed to simulate, depending on the alloy and according to the requirements profile and the calculated properties, a mechanical load on the workpiece to be manufactured and to add information concerning the simulated mechanical load to the simulation data.

3. The simulation system according to claim 1, wherein the analysis unit comprises a second simulation unit which is designed to simulate, depending on the alloy and according to the requirements profile and the information data, chemical properties of the workpiece to be manufactured and to add information concerning the simulated chemical properties to the simulation data.

4. The simulation system according to claim 1, wherein the information data specify information concerning at least one physical and/or chemical and/or mechanical property, in particular selected from:
thermal properties of the alloy,
media resistance of the alloy,
chemical properties,
amorphicity on the basis of a degree of contamination,
load-dependent aging phenomena and/or
cooling behavior of the alloy.

5. The simulation system according to claim 1, wherein the analysis unit is designed to make, according to the requirements profile, a preselection from alloys stored within the at least one memory unit and/or from manufacturing methods stored within the at least one memory unit, and a selection of an alloy and/or a manufacturing method takes place on the basis of the preselection.

6. The simulation system according to claim 1, wherein the analysis unit is designed
to generate a data pair by associating the selected alloy (L) and the selected manufacturing method (V) with the requirements profile (A),
to store the generated data pair in the at least one memory unit,
to specify an alloy associated with the requirements profile and a manufacturing method associated with the requirements profile when a requirements profile stored in the memory unit is input.

7. A method for selecting an alloy and a manufacturing method for a workpiece to be manufactured having amorphous properties, comprising the steps of:
inputting a requirements profile of the workpiece to be manufactured, in particular by means of an input unit, wherein the requirements profile specifies geometric and/or mechanical and/or chemical properties of the workpiece to be manufactured,
calculating mechanical and/or chemical and/or physical parameters, and comparing the calculated parameters with stored information data which contains information concerning physical and/or chemical and/or mechanical properties of a plurality of alloys for producing workpieces having amorphous properties,
simulating a plurality of workpieces according to the requirements profile and the information data,
creating simulation data on the basis of the simulations, and
assessing the simulated workpieces on the basis of the simulation data and the requirements profile,
selecting an alloy and a manufacturing method for the workpiece to be manufactured on the basis of the assessment,
outputting the selected alloy and the selected manufacturing method.

8. The method according to claim 7, wherein calculating properties of the workpiece to be manufactured which are specified by the requirements profile.

9. The method according to claim 7, wherein in that the information data specify information concerning at least one physical and/or chemical and/or mechanical properties, in particular selected from:
thermal properties of the alloy,
media resistance of the alloy,
chemical properties,
amorphicity on the basis of a degree of contamination,
load-dependent aging phenomena and/or
cooling behavior of the alloy.

10. The method according to claim 7, wherein
making a preselection from stored alloys and/or stored manufacturing methods according to the requirements profile,
selecting an alloy and/or a manufacturing method on the basis of the preselection.

11. The method according to claim 7, wherein
generating a data pair by associating the selected alloy and the selected manufacturing method with the requirements profile,
storing the generated data pair,
outputting an alloy associated with a stored requirements profile and a manufacturing method associated with the requirements profile if the requirements profile is already stored.

12. A computerreadable storage medium containing instructions that cause at least one processor to implement a method according to claim 7 when the method is executed by the at least one processor.

13. A manufacturing plant for manufacturing a workpiece having amorphous properties, comprising:
a simulation system for selecting an alloy and a manufacturing method for the workpiece to be manufactured according to, claim 1 and
a manufacturing unit which is designed to manufacture a workpiece using the simulation system.

14. The manufacturing plant according to claim 13,
wherein the manufacturing unit is designed as an injectionmolding device or as an additive manufacturing device.

15. A control method for controlling a manufacturing plant for manufacturing a workpiece having amorphous properties, for controlling a manufacturing plant according to, claim 13, wherein in that the manufacturing plant is operated with an alloy and a manufacturing method which are selected using a method comprising the steps of:
inputting a requirements profile of the workpiece to be manufactured, in particular by means of an input unit, wherein the requirements profile specifies geometric and/or mechanical and/or chemical properties of the workpiece to be manufactured,
calculating mechanical and/or chemical and/or physical parameters, and comparing the calculated parameters with stored information data which contains information concerning physical and/or chemical and/or mechanical properties of a plurality of alloys for producing workpieces having amorphous properties,
simulating a plurality of workpieces according to the requirements profile and the information data,
creating simulation data on the basis of the simulations, and
assessing the simulated workpieces on the basis of the simulation data and the requirements profile,
selecting an alloy and a manufacturing method for the workpiece to be manufactured on the basis of the assessment,
outputting the selected alloy and the selected manufacturing method.
